(19) United States
(12) Patent Application Publication
(10) Pub. No.: US 2008/0206291 A1
(43) Pub. Date: Aug. 28, 2008

(54) Polyurethane and Polyurea Microcapsules

(75) Inventors: Lahoussine Ouali, Vetraz-Monthoux (FR); Daniel Benczedi, Plan-Les-Ouates (CH)

Correspondence Address: WINSTON & STRAWN LLP;PATENT DEPARTMENT, 1700 K STREET, N.W., WASHINGTON, DC, 20006, US

(73) Assignee: FIRMENICH SA

(21) Appl. No.: 11/914,894

(22) PCT Filed: Jun. 30, 2006
(86) PCT No.: PCT/IB2006/052192

§ 371 Date: Nov. 19, 2007

(30) Foreign Application Priority Data
Jun. 30, 2005 (EP) 05105875.8

(51) Int. Cl. A61K 8/11; C11D 3/50; A61K 8/43; A61Q 5/02; A61K 8/87; A61Q 19/10; A61Q 5/12; C11D 17/08; A61Q 13/00; A61Q 15/00; C11D 9/44

(52) U.S. Cl. 424/401; 510/101; 510/107; 424/490; 424/65; 514/588; 512/4; 510/159; 510/119; 510/337; 510/527

(57) ABSTRACT

The present invention relates to a perfuming ingredient for body-care, detergents or softeners type consumer products that is made of microcapsules having a mean diameter of >1 to 500 μm with a guanidine-based polyurea or polyurea/polyurethane wall that contain a perfume and to the products that contain such perfuming ingredients and microspheres.

### TECHNICAL FIELD

The present invention relates to the use of specific perfume-containing microcapsules with a polyurea or a polyurea/polyurethane wall as perfuming ingredient in home or personal care products, as well as consumer products comprising surfactants and the microcapsules.

### BACKGROUND OF THE INVENTION AND PROBLEM TO BE SOLVED

The perfume industry's objective is to render everyone's life more pleasant by adding odoriferous compositions to consumer's products.

A first problem encountered in this task is the mere adherence of the perfume to the consumer product.

Moreover, a second problem lies also in the relatively rapid lost of the olfactive benefit provided by the odoriferous compounds due to their volatility, in particularly “top-notes”. This second problem is generally tackled using a delivery system, e.g. capsules containing a perfume, to release the fragrance in a controlled manner. However, said delivery systems may suffer from stability problems, in particular when incorporated into surfactants based products such as detergents, which are strongly aggressive towards said delivery systems.

WO 2004/098767 teaches about the use of microcapsules having polyurethane or polyurea wall and containing a perfume. Said document never mentions the possible use of guanidine as polyamine to form the capsule wall, but largely insists on a polyamine having a very different structure indicating even as preferred polyamine 1,6-hexamethylene diamine. However said document mentions the use of such microcapsule as perfuming ingredient into detergents or softeners.

Polyurea (guanidine based) microcapsules very similar to the ones disclosed in this invention are disclosed in US 2002/0198392. However said document indicates only that said capsules are suitable to perfume leather or textile by directly spraying a water dispersion of the microcapsule on the surface to be perfumed, i.e. the capsules are not deposited on the leather or textile via the use of a consumer product. Therefore said document differs from the present invention in that it does not disclose detergent, softeners or similar products containing the microcapsules (the water suspension described have totally different formulation) and does not allow to anticipate the unexpected stability and deposition performance of said capsules in the presence of high amount of surfactant.

Furthermore, even a combination of WO 2004/098767 and US 2002/0198392 would not allow a skilled person to anticipate the present invention's use and consumer product. U.S. Pat. No. 5,635,211 also mentions microcapsules similar to the ones of the present invention, but said capsules contain dyestuff and are used for no-carbon copy papers.

An object of the present invention is to provide perfumed consumer end products comprising surfactants and capable of providing improved olfactive properties to the surface treated with the consumer product, such as hair, skin or textiles.

### SUMMARY OF THE INVENTION

The present invention relates to the use as perfuming ingredient of specific polyurea or a polyurea/polyurethane microcapsules encapsulating a perfume. The invention concerns also the consumer products in the form of a home- or personal-care product, such as a detergent or a softener, and containing said capsules.

### DETAILED DESCRIPTION OF THE INVENTION

As first object, the present invention relates to the use, as perfuming ingredients in consumer products of the home- or personal-care type, of microcapsules having:
a mean diameter of >1 to 500 μm
a polyurea or a polyurea/polyurethane wall, which is the reaction product of the polymerisation between at least one polyisocyanate and at least one reactant selected from the group consisting of a water soluble guanidine salt, guanidine and mixture thereof with glycerine (i.e. 1,2,3-propanetriol), and wherein the polyisocyanates have at least two functional isocyanate groups; and
an encapsulated perfume.

In the present context we will refer to the “water soluble guanidine salt, guanidine and mixture thereof with glycerine (i.e. 1,2,3-propanetriol)” also as the “reactants” or “reactant”.

By “water soluble guanidine salt” it is meant a salt soluble in water and resulting from the reaction of guanidine with an acid. Examples of such salts are guanidine carbonate. Concerning the mixture that can be used as reactant, it is also useful to mention that according to some embodiments of the invention said mixtures may have a molar ratio (guanidine or guanidine salt)/glycerine of at least 0.2. According to another embodiment said ratio is at least 0.5. Alternatively, we can mention mixtures wherein the ratio is of about 1 or above. Guanidine and glycerine can be added to the suspension at the same time or successively.

In the present context, “mean diameter” refers to the arithmetic mean. The present inventors found that with the microcapsules of this size, optimal deposition and/or adherence of microcapsules to the targeted surface, e.g. textile, hair or skin, is obtained. Preferably, the microcapsules have a mean diameter of 2 to 50, more preferably 3 to 30 μm. The symbols “>1” mean that the diameter is larger than 1 μm.

In the present invention, a polyurea wall is the reaction product of a polyisocyanate with a guanidine or its salts. A polyurea/polyurethane wall is the reaction product of a polyisocyanates with mixture of glycerine and guanidine and/or its salts.

Microcapsules having a polyurea wall are preferred, due to their good stability in applications or in the consumer products.

The inventors found that the use of microcapsules having specific polyurea or polyurea/polyurethane wall is key in obtaining microcapsules that are at the fine balance between release and retention in a way that satisfactory slow and constant release of fragrances over time is achieved, once the capsules are placed on textiles or hair, while showing a good or improved stability (e.g. contrasts efficiently the extraction of the perfume by the surfactants of the consumer product). The use of guanidine or guanidine/glycerine mixture in the formation of the capsule walls is key in obtaining such effect, as will be shown in the examples further below.

Furthermore, the properties and stability of the capsules can be fine tuned by selecting polyisocyanates having a given number of reactive groups (i.e. isocyanate groups). According to a preferred embodiment of the invention, the polyisocyanate comprises 2, 3 or 4 isocyanate groups. Preferably it comprises 2 or 3 isocyanate groups.

According to a specific embodiment of the invention said polyisocyanate comprises a 1,6- or a 1,4-diisocyanate moiety. In particular one may cite polyisocyanates that are derivatives of hexamethylene or of isophorone. Examples of suitable polyisocyanates are isophorone diisocyanate, hexamethylene diisocyanate, trimer of hexamethylene diisocyanate (Desmodur®N3300, Bayer), trimer of isophorone diisocyanate (Desmodur®Z4470BA), Biuret of hexamethylene diisocyante (Desmodur®N100, Desmodur®N75BA, Bayer).

Following the above numbers of functional groups, an optimal reticulation or network of the capsules wall is achieved, providing thus microcapsules exhibiting a surprising prolonged slow release of fragrances, as well as a surprising improved stability in the consumer product.

The perfume encapsulated can be in the form of a single perfuming ingredient or of an admixture thereof (i.e. a perfuming composition). Said perfuming ingredient may be of synthetic or natural origin.

Specific examples of such perfuming ingredient may be found in the current literature, for example in Perfume and Flavour Chemicals, 1969 (and later editions), by S. Arctander, Montclair N.J. (USA). They are well known to the skilled person in the art of perfuming consumer products, that is, of imparting an odour to a consumer product.

Preferably, the perfume does not contain primary alcohols, as these compounds may react with the polyisocyanates during the wall-formation process. Furthermore, said perfume contains less than 10% of its own weight of secondary and tertiary alcohols.

Preferably, perfuming ingredients have 4 to 20 carbon atoms. These compounds are capable of slowly defusing through the wall of the capsules of the present invention.

As non limiting examples of suitable perfuming ingredients one may cite the ones selected from the group of esters, lactones, ketones, ethers or, optionally, the tertiary or secondary alcohols and which are of current use in the perfumery industry. Yet, more particularly one may cite:
the damascones, such as delta damascone;
enones, such as 1-(5,5-dimethyl-1-cyclohexen-1-yl)-4-penten-1-one;
ketones, such as methyl dihydrojasmonate or (1′R)-2-[2-(4′-methyl-3′-cyclohexen-1′-yl)propyl]cyclopentanone;
esters or lactones, such as 2,2,2-trichloro-1-phenylethyl acetate, methyl dihydro-jasmonate or pentadecenolide; or
ethers, such as dodecahydro-3a,6,6,9a-tetramethyl-naphtho[2,1-b]furan.

According to a preferred embodiment of the invention, the capsule may contain from 60% to 98% of perfume, relative to their total weight. Alternatively they may contain from 85% to 95% of perfume.

The microcapsules of the present invention are preferably prepared, in a first step, by preparing an emulsion or dispersion comprising a hydrophobic solution, a water phase, polyisocyanates, and the reactant, and wherein the droplet size is comprised between >1 and 500 μm.

Preferably, the hydrophobic solution may be prepared by mixing the perfume with hydrophobic solvents of current use in the perfume industry, which are preferably not alcohols. Examples of such solvents are diethyl phthalate, isopropyl myristate, benzyl benzoate, ethyl citrate, limonene or other terpenes, or isoparaffins.

Preferably, the hydrophobic solution comprises less than 30 wt. % of solvent. More preferably, the hydrophobic solution comprises less than 20%, and even more preferably less than 10 wt % of solvent. Most preferably, the hydrophobic solution consists essentially of perfume and is essentially free of a solvent.

Preferably, the emulsion or dispersion comprises about 10-50 wt. % of perfume, more preferably 20-40 wt. % of perfume.

The polyisocyanate may then be added to the hydrophobic solution. Preferably, the polyisocyanates are added at a percentage of 2 to 20 wt. % of the hydrophobic solution. The emulsion or dispersion may be prepared by high shear mixing and adjusted to the desired droplet size. Droplet size may be checked with light scattering measurements or microscopy.

For the purpose of the present invention, an emulsion is characterized by the stabilization of the oil droplets by emulsifiers, while in a dispersion the droplets are generally stabilized by a colloidal stabilizer. Accordingly, an emulsifier and/or a colloidal stabilizer is preferably added to the emulsion or dispersion. Examples of colloidal stabilizers are polyvinyl alcohol, cellulose derivatives such hydroxyethyl cellulose, polyethylene oxide, copolymers of polyethylene oxide and polyethylene or polypropylene oxide, or copolymers of acrylamide and acrylic acid.

Examples of emulsifier are anionic surfactant such as sodium dodecyl sulfate or Stepantex®, non ionic surfactant such as diblock copolymers of polyethylene oxide and polyethylene or polypropylene oxide.

At this stage, the reactant (e.g. guanidine carbonate) may be added, preferably after mixing with water. Preferably, for each mole of isocyanate present in the hydrophobic solution 1 to 3, preferably 1.2 to 2 moles of primary amine groups (in reactant) are added to the emulsion. Accordingly, the emulsion or dispersion preferably comprises an excess of reactant.

In a further step, a polymerisation between the polyisocyanates and the reactant in the emulsion or dispersion is induced. In the case of guanidine or its salts, no specific action is required for inducing the polymerisation, because the reaction may start immediately after adding the polyamines to the aqueous solution. In the case of mixture comprising glycerine, the polymerisation may be helped by slowly heating, preferably to 40-90° C. in 0.5 to 5 hours. In addition or alternatively, polymerisation may be induced by addition of a catalyst. An example for a suitable catalyst is 1,4-diazabicyclo[2,2,2]octane.

Preferably, the reaction is maintained for 2 to 30, preferably 5 to 20 hours.

As mentioned above, the microcapsules described can be used as perfuming ingredients in consumer products of the home- or personal-care type. This result is highly surprising since said consumer products contain high amounts (typically more than 10% of their own weight) of specific type of surfactant/tensioactive/solvents and which are known to significantly diminish the stability and the performance of said capsules. As shown in the examples further below, the use of the capsule mentioned above provides improved deposition of the perfume on the treated surface together with an improved stability in a chemically aggressive environment. In other word the use of said capsules in the above-mentioned applications provides unforeseeable advantages over the same use of other similar prior art capsules.

Therefore another object of the present invention is a consumer product, in the form of a home- or personal-care product, and comprising the capsules of the invention. Said consumer product may be a solid or a liquid product. According to a particular embodiment, liquid products are preferred.

The expression “home- or personal-care” has here the usual meaning in the art, and in particular it includes products such as body-care, hair-care or home-care products. Examples of liquid products according to the invention may be selected from the group consisting of a shampoo or a hair conditioner, a liquid detergent, a fabric softener, a shower or bath mousse, oil or gel, a deodorant or an antiperspirant. Preferably, the liquid perfumed product is a shampoo, a liquid detergent or a fabric softener.

Examples of solid products according to the invention may be selected from the group consisting of a soap bar, a powder detergent or an air-freshener.

As detergents there are considered applications such as detergent compositions or cleaning products for washing up or for cleaning various surfaces, for example, intended for textiles, dishes or hard surfaces (floors, tiles, stone-floors, etc). Preferably, the surface is a textile.

Conveniently, the reaction mixture may be used as such to perfume the consumer products. For example, the reaction mixture (containing about 20-40 wt. % perfume) may be directly added to a liquid fabric softener at a rate of 0.1-30 wt. %, resulting in a total perfume content of about 0.0333-10 wt. % in the fabric softener. Preferably, a consumer product according to the invention comprises about 0.01 to 4 wt. %, or even 4.5%, of it own weight, in capsules as defined above and containing the perfume.

Of course, the above concentration may be adapted according to the olfactive effect desired in each product.

Similarly, the reaction mixture comprising the microcapsules of the invention may be sprayed onto a dry, powdered product, such as a washing powder or powdered detergent.

### BRIEF DESCRIPTION OF THE DRAWINGS

FIG. 1 shows perfume intensity over time of towels washed with a softener (see example 4) containing a free perfume (pure α-Damascone), or containing the same perfume encapsulated in the polyurea microcapsules obtained in example 2 (capsule example 2), or containing the same perfume encapsulated in the polyurethane microcapsules obtained in example 1, i.e. not according to the invention (capsule example 1), respectively. Guanidine-based polyurea capsule show higher perfume intensity over the entire time period (7 days) (1: no odor, 2: weak odor, 3: slightly weak odor, 4: medium odor, 5: slightly strong odor, 6: strong odor, 7: very strong odor).

FIG. 2 shows perfume intensity over time of towels washed with a softener (see example 4) containing a free perfume (pure α-Damascone), or containing the same perfume encapsulated in the polyurea microcapsules obtained in example 3 (capsule example 3), ethyl 2-methylpentanoate was used as perfume, and that perfume intensity was evaluated at day 1 and day 3. (1: no odor, 2: weak odor, 3: slightly weak odor, 4: medium odor, 5: slightly strong odor, 6: strong odor, 7: very strong odor).

FIG. 3 show the percentage of licking from a microcapsule (polyurea or polyurethane walls), of each component of a complex perfuming composition into the bulk of a softener base. The microcapsule used have guanidine-based polyurea walls, according to the invention, or glycerine-based polyurea walls, not according to the invention, and are compared to a softener wherein the same free perfume has been introduced.

### EXAMPLES

### Example 1

### Encapsulation of a Single Perfuming Ingredient in Polyurethane Capsules

### Synthesis of Polyurethane Capsules:

| Chemicals | Amounts | |
|---|---|---|
| Desmodur N3300 ⁽¹⁾ | 2.10 | g |
| Perfume (α-Damascone) ⁽²⁾ | 30.00 | g |
| Propantriol | 0.27 | g |
| Celvol 523 ⁽³⁾, 1% in water | 70.00 | g |
| Deionised water | 4.77 | g |
| DABCO ⁽⁴⁾ | 0.02 | g |
| Total | 107.16 | g |

⁽¹⁾ polyisocyanate, Bayer
⁽²⁾ (+−)-(E)-1-(2,6,6-trimethyl-2-cyclohexen-1-yl)-2-buten-1-one
⁽³⁾ polyvinyl alcohol, Celanese Chemicals
⁽⁴⁾ 1,4-Diazabicyclo[2.2.2]octane, Fluka

The stabilizer solution (water+Celvol) with the oil phase (hydrophobic solution comprising the perfume and the polyisocyanate) is stirred for 1 minute on magnetic stirrer. The oil phase is then emulsified by ultra-turrax for 4 minutes when the temperature is maintained at 25° C. The size distribution is controlled by light scattering measurements and revealed that the mean diameter of oil droplets is about 9 μm. The emulsion is transferred to the reactor. Propantriol is mixed with water and added gradually to the emulsion (during 3 min). The suspension is then heated from 25° C. to 70° C. during 2 hours. The reaction mixture is kept at 70° C. for 16 hours. At the end of the polymerization process, the reaction mixture is cooled down. The resulting capsules suspension is then characterized by determining its solid content and size distribution. The solid content is determined to be 32.4 wt. % (including the perfume; perfume contents of the capsules: 85-88%) by heating 1 g of the final capsule suspension at 50° C. for a duration comprising between 3 to 7 hours, mean size of capsules suspension was 9 μm.

### Example 2

### Encapsulation of Single Perfuming Ingredient in Polyurea Capsules

| Chemicals | Amounts | |
|---|---|---|
| Desmodur N3300 ⁽¹⁾ | 2.10 | g |
| Perfume (α-Damascone) | 30.00 | g |
| Guanidine carbonate | 0.53 | g |
| Elvanol 52-22 ⁽²⁾, 1% in water | 70.00 | g |
| Deionised water | 4.77 | g |
| Total | 107.40 | g |

⁽¹⁾ polyisocyanate, Bayer
⁽²⁾ polyvinyl alcohol, Celanese Chemicals

The polyisocyanate is mixed with perfume and added to the stabilizer solution (water and Elvanol). The obtained mixture is homogenized with Ultra-turax for 4 minutes at room temperature then transferred to a reactor equipped with mechanical stirring system. The shear rate is fixed at 200 rpm. The guanidine carbonate is dissolved in deionized water and added dropwise to reactor while stirring. The reaction mixture is heated to 70° C. during 2 hours. At the end of polymerization process, the reaction mixture is cooled down at room temperature. The resulting capsules suspension is then characterized by determining its solid content and size distribution. The mean size of capsules suspension was 8.1 μm while the solid content (determinate as in Example 1) was 32.7% (including the perfume; perfume contents of the capsules: 85-88%).

### Example 3

### Encapsulation of Single Perfuming Ingredient in Polyurea Capsules

Example 2 was repeated with ethyl 2-methylpentanoate as encapsulated perfume. The characterization of the capsules thus obtained gave very similar results as for the ones of Example 1.

### Example 4

### Evaluation of the Delivery Systems

A concentrated fabric softener base was prepared by admixing the following ingredients:

| Ingredient | Parts by weight |
|---|---|
| Stepantex ® VS 90 diester quat⁽⁴⁾ | 16.5 |
| Calcium chloride | 0.2 |
| Deionised water | 82.3 |
| Total | 100.0 |

⁽⁴⁾origin: Stepan Europe, France

The delivery systems of Examples 1 and 2 were evaluated in a softener application. Accordingly, the aqueous suspensions obtained in examples 1 and 2 were directly added at 1 mmole perfume for 36 g of fabric softener base each. After a vigorous stirring the mixtures were poured into the fabric softener compartment of a Miele Novotronic W300-33 CH washing machine. 85 g of un-perfumed detergent was poured into the detergent compartment. Then, 15 cotton towels (18 cm*18 cm, about 30 g each) and 2 kg of large cotton towels (8 towels of 50 cm*100 cm) were washed at 40° C. using the short cycle program. At the end of the wash, the towels were dried in a drying room for 24 hours and then packed in aluminium foil and evaluated by a panel of 20 persons at 1 day, 3 days and 7 days after the wash.

Each panelist was asked to rate the various towels on an intensity scale of 1 to 7 (1: no odor, 2: weak odor, 3: slightly weak odor, 4: medium odor, 5: slightly strong odor, 6: strong odor, 7: very strong odor)

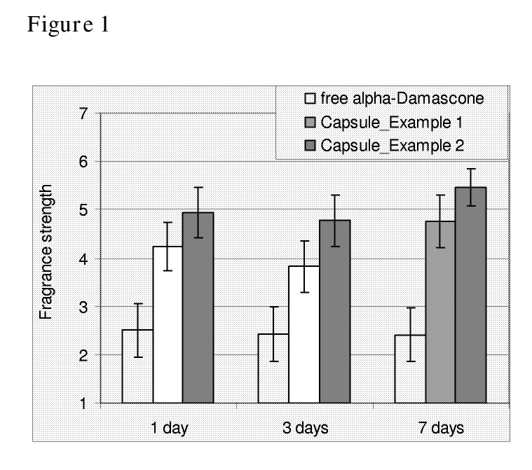

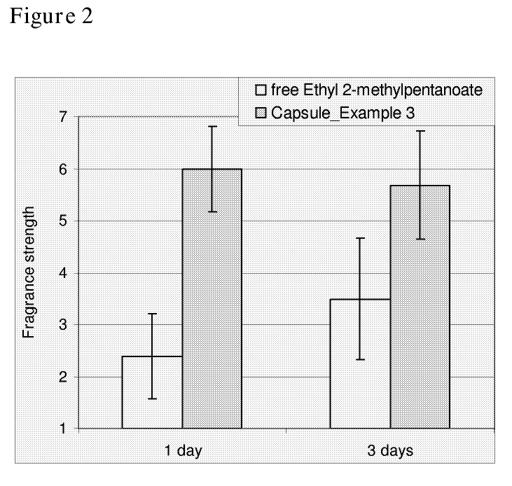

As reference a fabric softener base containing 1 mmole of pure alpha-damascone was used, which was tested in the same way. The results are shown in FIG. 1 and FIG. 2. As can be seen from FIG. 2, the invention's softener is able to deliver an olfactive benefit by far superior to the simple use of a free perfume. Furthermore, as can be seen from FIG. 1, softeners having a capsule with walls based on guanidine perform significantly better than softeners having capsules with walls based on pure glycerine, i.e. outside the scope of the present invention. The improved performances are observable over a prolonged period of time, e.g. 1, 3 or even 7 days.

### Example 5

### Encapsulation of a Perfuming Composition in Polyurea Capsules

It was prepared a perfuming composition comprising the following ingredients: delta damascone (A), 1-(5,5-dimethyl-1-cyclohexen-1-yl)-4-penten-1-one (Neobutenone®) (B), 2-ethoxynaphthalene (C), 2,2,2-trichloro-1-phenylethyl acetate (D), methyl dihydrojasmonate (Hedione®) (E), dodecahydro-3a,6,6,9a-tetramethyl-naphtho[2,1-b]furan (Cetalox®) (F), (1′R)-2-[2-(4′-methyl-3′-cyclohexen-1′-yl)propyl]cyclopentanone (G), pentadecenolide (Habanolide®) (H), isopropyl myristate (I).

This perfume was encapsulated according to the same protocol described in Example 2 above, providing thus microcapsules according to the invention. The mean size of capsules suspension was 12.4 μm while the solid content was 32%.

### Example 6

### Encapsulation of a Perfuming Composition in Polyurethane Capsules

The same perfume of Example 5 was encapsulated according the same protocol described in Example 1 above, providing thus polyurethane microcapsules. The mean size of capsules suspension was 11.8 μm while the solid content was 32%.

### Example 7

### Stability of the Microcapsules when Incorporated into a Consumer Product of the Softener Type

Following the protocol of Example 4, various softeners containing respectively the free perfume (reference softener), the perfume encapsulated in microcapsules according to the invention (Example 5) and the perfume encapsulated in microcapsules not according to the invention (Example 6), have been prepared.

Each softener has been stored at 38° C. for a period of 2 to 4 weeks, and then the free perfume is extracted with a mixture solvent composed of iso-octane and ether (90/10, w/w). Practically, 4 g of softener containing perfume or encapsulated perfume are mixed with 10 ml of solvent. The final mixture is maintained under stirring for 30 min. After phase separation, the organic phase is analyzed by GC-MS to quantify the amount of free perfume.

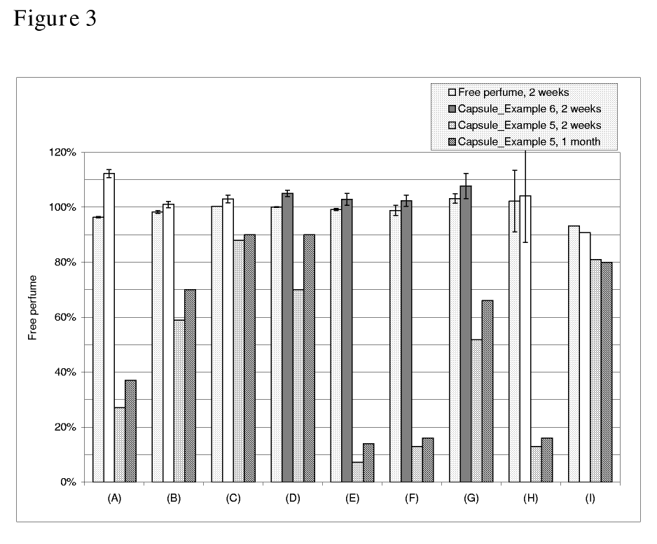

FIG. 3 shows the results obtained. Comparing the performance of each capsule it is visible that microcapsule according to Example 5 shows an improved stability compared to the one of the microcapsules according to Example 6 (i.e. a better retention of each perfuming ingredient or if preferred a lower licking). This shows that the consumer products having microcapsules according to the invention are able to deliver a higher amount of perfume, and consequently a higher benefit, to the surface treated (e.g. a textile). This fact is confirmed by the test performed in Example 4.

When the amount of free perfume present in the bulk is compared to the one of the reference softeners then it is visible that the use of the microcapsule according to the present invention allows to maintain still 57% of the perfume in the microcapsule, while the use of microcapsule not according to the invention, although very similar to the latter, provides no benefit at all (all the perfume is in the bulk, i.e. not encapsulated).

1-12. (canceled)

13. A home- or personal-care consumer product containing, as a perfuming ingredient, microcapsules that encapsulate a perfume and that have a mean diameter of >1 to 500 μm and a polyurea or a polyurea/polyurethane wall which is the reaction product of polymerisation between at least one polyisocyanate, glycerine (i.e. 1,2,3-propanetriol) and at least one reactant selected from the group consisting of a water soluble guanidine salt, guanidine and mixtures thereof; wherein the polyisocyanate has at least two functional isocyanate groups.

14. The product according to claim 1, wherein the polyisocyanate is isophorone diisocyanate, hexamethylene diisocyanate, trimer of hexamethylene diisocyanate, trimer of isophorone diisocyanate, or Biuret of hexamethylene diisocyante.

15. The product according to claim 1, wherein the reactant is guanidine or guanidine carbonate.

16. The product according to claim 1, wherein the microcapsules have a mean diameter of 2 to 50 μm.

17. The product according to claim 1, wherein the perfume does not contain primary alcohols, and contains less than 10% of its own weight of secondary and tertiary alcohols.

18. The product according to claim 1, wherein the perfume contains perfuming ingredients selected from the group of esters, lactones, ketones, ethers or, optionally, the tertiary or secondary alcohols and which are of current use in the perfumery industry.

19. The product according to claim 1, in the form of a body-care, hair-care or home-care product.

20. The product according to claim 19, which is a liquid product and is in the form of a shampoo or a hair conditioner, a liquid detergent, a fabric softener, a shower or bath mousse, oil or gel, a deodorant or an antiperspirant.

21. The product according to claim 19, which is in the form of a shampoo, a liquid detergent or a fabric softener.

22. The product according to claim 19, which is a solid product and is in the form of a shampoo, a liquid detergent or a fabric softener.

23. A method for making a home- or personal-care consumer product, which comprises:
preparing a perfuming ingredient by forming microcapsules that encapsulate a perfume; wherein the microcapsules are formed to have a mean diameter of >1 to 500 μm, with the forming comprising polymerising at least one polyisocyanate having at least two functional isocyanate groups, glycerine (i.e. 1,2,3-propanetriol) and at least one reactant selected from the group consisting of a water soluble guanidine salt, guanidine and mixtures thereof to form as a reaction product a polyurea or a polyurea/polyurethane wall for the microcapsules which wall encapsulates the perfume; and
incorporating the microcapsules into the consumer product to provide perfuming characteristics thereto.

24. Microcapsules that encapsulate a perfume and that have a mean diameter of >1 to 500 μm and a polyurea or a polyurea/polyurethane wall which is the reaction product of polymerisation between at least one polyisocyanate, glycerine (i.e. 1,2,3-propanetriol) and at least one reactant selected from the group consisting of a water soluble guanidine salt, guanidine and mixtures thereof; wherein the polyisocyanate has at least two functional isocyanate groups.

25. A method for preparing a perfuming ingredient in the form of microcapsules that encapsulate a perfume which comprises providing a perfume and polymerising at least one polyisocyanate having at least two functional isocyanate groups, glycerine (i.e. 1,2,3-propanetriol) and at least one reactant selected from the group consisting of a water soluble guanidine salt, guanidine and mixtures thereof to form as a reaction product a polyurea or a polyurea/polyurethane wall for the microcapsules which wall encapsulates the perfume to form the microcapsules; wherein the microcapsules are formed to have a mean diameter of >1 to 500 μm.

26. A perfuming ingredient comprising microcapsules obtained by the method of claim 25.

27. A perfuming ingredient comprising microcapsules obtainable by the method of claim 25.
